(19) United States
(12) Patent Application Publication
(10) Pub. No.: US 2021/0147777 A1
(43) Pub. Date: May 20, 2021

(54) CO-CULTURE DEVICE AND CO-CULTURE METHOD

(71) Applicants: SHIMADZU CORPORATION, Kyoto-Shi (JP); TOKYO INSTITUTE OF TECHNOLOGY, Tokyo (JP)

(72) Inventors: Yoichi FUJIYAMA, Kyoto-Shi (JP); Hiroomi GOTO, Kyoto-Shi (JP); Masaki KANAI, Kyoto-Shi (JP); Yoh-ichi TAGAWA, Tokyo (JP)

(73) Assignees: SHIMADZU CORPORATION, Kyoto -shi (JP); TOKYO INSTITUTE OF TECHNOLOGY, Tokyo (JP)

(21) Appl. No.: 16/951,087

(22) Filed: Nov. 18, 2020

(30) Foreign Application Priority Data
Nov. 20, 2019 (JP) 2019-209694

(51) Int. Cl. C12M 3/06; C12M 1/34; B01L 3/00; C12N 1/04; C12N 1/20

(52) U.S. Cl. CPC C12M 23/16; C12M 41/34; B01L 2300/0864; C12N 1/04; C12N 1/20; B01L 3/502715

(57) ABSTRACT

A co-culture device includes: a first body including a first membrane having a first main surface for culturing cells and a second main surface opposite to the first main surface, a first flow path partially defined by the first main surface, the first flow path configured for a first culture medium to flow therethrough, and a second flow path partially defined by the second main surface, the second flow path configured for a second culture medium having a higher dissolved oxygen concentration than that of the first culture medium to flow therethrough; and an oxygen concentration adjuster for adjusting the dissolved oxygen concentration in the first culture medium to be supplied to the first flow path.

### BACKGROUND OF THE INVENTION

### Field of the Invention

The present invention relates to a co-culture device and a co-culture method.

### Description of the Background Art

Development of a device that simulates the intestinal environment and the like has been promoted for the purpose of studying drug kinetics, drug metabolism and the like. WO 2018/079793 discloses a system that simulates the intestinal environment by disposing, in an anaerobic chamber, a device having intestinal epithelial cells seeded on a porous membrane.

### SUMMARY OF THE INVENTION

In studying drug kinetics, drug metabolism and the like, oxygen concentrations are different even in the small intestine, for example, between the side closer to the large intestine and the side farther from the large intestine. Conventional techniques including WO 2018/079793 do not contemplate simulating an environment having different oxygen concentrations with one system.

The present invention provides a co-culture device and a co-culture method capable of simulating an environment having different oxygen concentrations.

A co-culture device of the present invention includes: a first body including a first membrane having a first main surface for culturing cells and a second main surface opposite to the first main surface, a first flow path partially defined by the first main surface, the first flow path configured for a first culture medium to flow therethrough, and a second flow path partially defined by the second main surface, the second flow path configured for a second culture medium having a higher dissolved oxygen concentration than that of the first culture medium to flow therethrough; and an oxygen concentration adjuster for adjusting the dissolved oxygen concentration in the first culture medium to be supplied to the first flow path.

A co-culture method of the present invention includes: adjusting a dissolved oxygen concentration in a first culture medium; passing the first culture medium having the adjusted dissolved oxygen concentration through a first flow path partially defined by a first main surface of a first membrane on which cells are cultured; and passing a second culture medium having a higher dissolved oxygen concentration than that of the first culture medium through a second flow path partially defined by a second main surface of the first membrane opposite to the first main surface, and supplying oxygen from the second culture medium toward the first flow path through the first membrane.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a schematic cross-sectional view of a co-culture device 100.

FIG. 2 is an exploded perspective view of a first body 10.

FIG. 3 is a flowchart of a co-culture method according to a first embodiment.

FIG. 4 is a schematic cross-sectional view of a co-culture device 200.

### DESCRIPTION OF THE PREFERRED EMBODIMENTS

Embodiments will be described in detail with reference to the drawings. The same or corresponding parts are designated by the same reference characters in the drawings below, and redundant description will not be repeated.

### First Embodiment

The configuration of a co-culture device according to a first embodiment (hereinafter referred to as “co-culture device 100”) is described below.

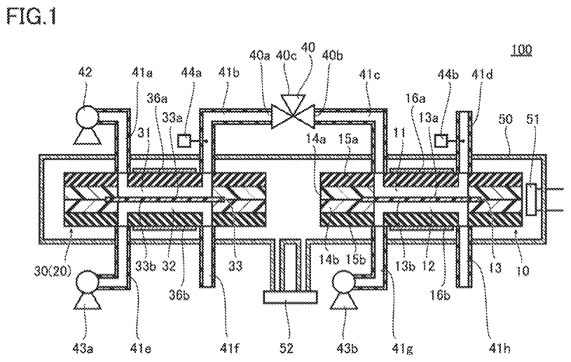

FIG. 1 is a schematic cross-sectional view of co-culture device 100. As shown in FIG. 1, co-culture device 100 includes a first body 10 and an oxygen concentration adjuster 20.

First body 10 includes therein a first flow path 11, a second flow path 12, and a first membrane 13. A first culture medium flows through first flow path 11. A second culture medium flows through second flow path 12.

First membrane 13 has a first main surface 13a and a second main surface 13b. Cells are cultured on first main surface 13a. These cells are intestinal epithelial cells that form tight junctions on first main surface 13a, for example. A specific example of these cells is Caco-2 cells. First membrane 13 is a track-etched membrane made of polycarbonate, for example. First membrane 13 may be a collagen vitrigel membrane, for example. First membrane 13 is not particularly limited so long as cell culture is possible on first main surface 13a, and oxygen is transmitted through first membrane 13. Second main surface 13b is opposite to first main surface 13a.

First flow path 11 is partially defined by first main surface 13a. Second flow path 12 is partially defined by second main surface 13b.

The first culture medium may contain bacteria. The bacteria contained in the first culture medium are enterobacteria such as Escherichia coli. However, the bacteria contained in the first culture medium are not limited as such. The first culture medium may contain a component other than the bacteria instead of or in addition to the bacteria. The second culture medium has a higher dissolved oxygen concentration than that of the first culture medium.

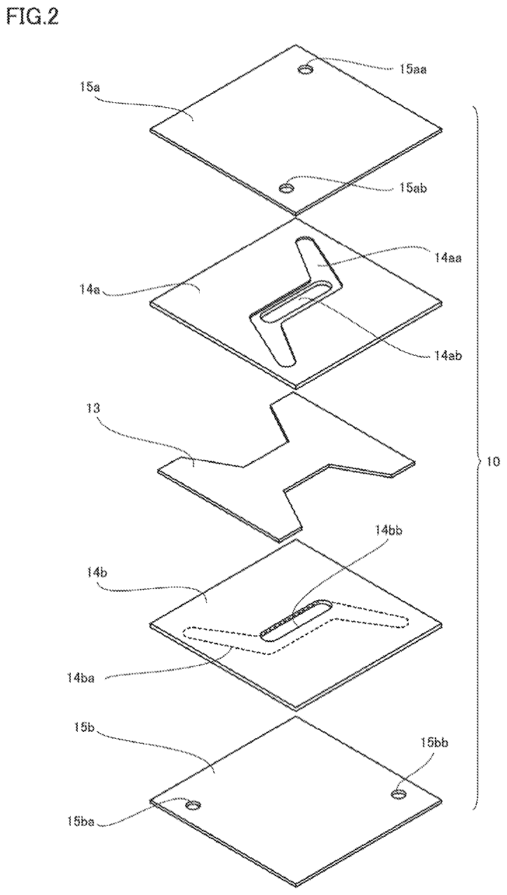

FIG. 2 is an exploded perspective view of first body 10. As shown in FIG. 2, first body 10 is formed of, for example, a first sheet 14a and a second sheet 14b, a first glass plate 15a and a second glass plate 15b, and first membrane 13.

First sheet 14a and second sheet 14b are bonded together. First sheet 14a has a groove 14aa and a hole 14ab formed therein. Groove 14aa is formed in a surface of first sheet 14a opposite to the surface that is bonded to second sheet 14b. Hole 14ab extends through first sheet 14a in a thickness direction. Groove 14aa is connected to hole 14ab.

Second sheet 14b has a groove 14ba and a hole 14bb formed therein. Groove 14ba is formed in a surface of second sheet 14b opposite to the surface that is bonded to first sheet 14a. Hole 14bb extends through second sheet 14b in the thickness direction. Hole 14bb is formed at a position coinciding with hole 14ab. Groove 14ba is connected to hole 14bb. First membrane 13 is sandwiched between first sheet 14a and second sheet 14b.

First sheet 14a and second sheet 14b bonded together are sandwiched between first glass plate 15a and second glass plate 15b. First glass plate 15a is bonded to a surface of first sheet 14a opposite to the surface that is bonded to second sheet 14b. Second glass plate 15b is bonded to a surface of second sheet 14b opposite to the surface that is bonded to first sheet 14a.

The bonding of first sheet 14a and second sheet 14b, the bonding of first sheet 14a and first glass plate 15a, and the bonding of second sheet 14b and second glass plate 15b are performed, for example, by compression bonding while the surfaces are activated with oxygen plasma.

A space defined by first membrane 13, groove 14aa, hole 14ab and first glass plate 15a serves as first flow path 11, and a space defined by first membrane 13, groove 14ba, hole 14bb and second glass plate 15b serves as second flow path 12.

First glass plate 15a has a hole 15aa and a hole 15ab formed therein, which extend through first glass plate 15a in the thickness direction and communicate with first flow path 11. Hole 15aa serves as an inlet of first flow path 11. Hole 15ab serves as an outlet of first flow path 11. Second glass plate 15b has a hole 15ba and a hole 15bb formed therein, which extend through second glass plate 15b in the thickness direction and communicate with second flow path 12. Hole 15ba serves as an inlet of second flow path 12. Hole 15bb serves as an outlet of second flow path 12.

First sheet 14a and second sheet 14b are made of a material having low gas permeability. First sheet 14a and second sheet 14b are made of silicone rubber, for example. First glass plate 15a and second glass plate 15b also have low gas permeability, making it unlikely for oxygen to enter first flow path 11 and second flow path 12 from outside of first body 10.

As shown in FIG. 1, first body 10 may further include an electrode 16a and an electrode 16b. Electrode 16a is formed on first glass plate 15a, and electrode 16b is formed on second glass plate 15b. Electrode 16a and electrode 16b are made of platinum (Pt), for example. Electrode 16a and electrode 16b can be formed by sputtering, for example.

An electrical resistance value between first flow path 11 and second flow path 12 can be measured by application of a voltage to electrode 16a and electrode 16b. This electrical resistance value increases when the cells cultured on first main surface 13a form tight junctions, and decreases when the tight junctions are not formed. By measuring this electrical resistance value, therefore, it can be determined whether the cells cultured on first main surface 13a are forming tight junctions or not (whether the cells have been damaged or not). Note that this electrical resistance value is measured with a four-terminal method, for example.

Oxygen concentration adjuster 20 is a second body 30, for example. Second body 30 has a similar configuration to that of first body 10. That is, second body 30 includes a third flow path 31, a fourth flow path 32, and a second membrane 33.

The first culture medium flows through third flow path 31. A third culture medium flows through fourth flow path 32. The third culture medium has a higher dissolved oxygen concentration than that of the first culture medium. The third culture medium is the same as the second culture medium, for example.

Second membrane 33 has a third main surface 33a and a fourth main surface 33b. Cells are cultured on third main surface 33a. The cells cultured on third main surface 33a may be the same as or different from the cells cultured on first main surface 13a. Second membrane 33 should only be such that cell culture is possible on third main surface 33a, and oxygen is transmitted through second membrane 33. Second membrane 33 may be the same as or different from first membrane 13.

Third flow path 31 is partially defined by third main surface 33a. Fourth flow path 32 is partially defined by fourth main surface 33b.

Second body 30 may further include an electrode 36a and an electrode 36b for measuring an electrical resistance value between third flow path 31 and fourth flow path 32.

Co-culture device 100 further includes a three-way connector 40, a tube 41a to a tube 41h, a pump 42, pumps 43a and 43b, and an oxygen sensor 44a and an oxygen sensor 44b.

Three-way connector 40 has a first connection port 40s, a second connection port 40b, and a third connection port 40c. Although not shown, a septum is connected to third connection port 40c. Tube 41a to tube 41h are made of a material having low gas permeability. Tube 41a to tube 41h are made of PEEK (polyether ether ketone) resin, for example. Pump 42, pump 43a and pump 43b are each a syringe pump, for example.

Tube 41a is connected to an inlet of third flow path 31. Tube 41b connects an outlet of third flow path 31 to first connection port 40a. Tube 41c connects second connection port 40b to the inlet of first flow path 11. Tube 41d is connected to the outlet of first flow path 11.

Tube 41e is connected to an inlet of fourth flow path 32. Tube 41f is connected to an outlet of fourth flow path 32. Tube 41g is connected to the inlet of second flow path 12. Tube 41h is connected to the outlet of second flow path 12. Oxygen sensor 44a and oxygen sensor 44b are attached to tube 41b and tube 41d, respectively. The dissolved oxygen concentrations in the first culture medium that has flowed through third flow path 31 and first flow path 11 are thereby monitored.

By driving pump 42, the first culture medium is supplied through tube 41a to third flow path 31. During the flow of the first culture medium through third flow path 31, oxygen contained in the first culture medium is consumed by the bacteria in the first culture medium and the cells cultured on third main surface 33a. Thus, first culture medium decreases in dissolved oxygen concentration by passing through third flow path 31.

The first culture medium that has flowed through third flow path 31 flows into three-way connector 40 through first connection port 40a via tube 41b, and flows out through second connection port 40b. The first culture medium that has flowed out through second connection port 40b is supplied to first flow path 11 via tube 41c. Thus, the dissolved oxygen concentration in the first culture medium to be supplied to first flow path 11 is adjusted by second body 30 (oxygen concentration adjuster 20). The first culture medium that has flowed through third flow path 31 may partially be collected by the septum. To the first culture medium that has flowed through third flow path 31, bacteria may be added from the septum before the first culture medium is supplied to first flow path 11.

By driving pump 43a, the second culture medium is supplied through tube 41c to fourth flow path 32. By driving pump 43b, the second culture medium is supplied through tube 41g to second flow path 12.

During the passage of the second culture medium through second flow path 12, oxygen in the second culture medium is supplied through first membrane 13 to the cells cultured on first main surface 13a. During the passage of the third culture medium through fourth flow path 32, oxygen in the third culture medium is supplied through second membrane 33 to the cells cultured on third main surface 33a. Thus, the cells cultured on first main surface 13a am maintained irrespective of the dissolved oxygen concentration in the first culture medium, and the cells cultured on third main surface 33a are maintained irrespective of the dissolved oxygen concentration in the third culture medium.

Co-culture device 100 may further include a container 50, a heater 51, and a degasser 52. Liquid is stored in container 50. This liquid is Fluorinert®, for example. First body 10 and second body 30 are immersed in this liquid.

Heater 51 maintains a temperature of the liquid stored in container 50 by heating the liquid stored in container 50. Degasser 52 degases oxygen contained in the liquid stored in container 50.

Degasser 52 includes a chamber, a tube disposed within the chamber, and a first pump and a second pump, for example. By driving the first pump, the liquid stored in container 50 circulates between container 50 and degasser 52. During the circulation, the liquid stored in container 50 flows through the tube. By driving the second pump, the interior of the chamber is evacuated. The tube is made of a gas permeable material. The liquid stored in container 50 is thereby degassed while flowing through the tube.

Though the example above has been described with respect to the case where there is one first body 10, there may be two or more first bodies 10. When there are two or more first bodies 10, first body 10 other than first body 10 connected to second body 30 is connected in series. One first body 10 connected to the upstream side of another first body 10 functions as oxygen concentration adjuster 20 for the another first body 10 connected to the downstream side of the one first body 10.

A co-culture method according to the first embodiment will be described below.

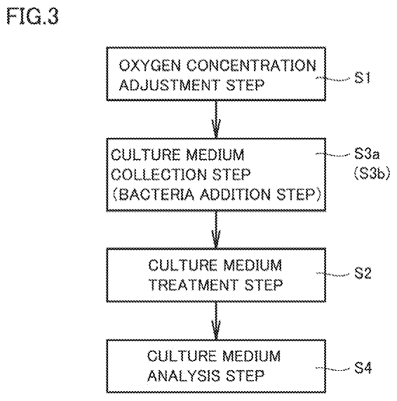

FIG. 3 is a flowchart of the co-culture method according to the first embodiment. As shown in FIG. 3, the co-culture method according to the first embodiment includes an oxygen concentration adjustment step S1 and a culture medium treatment step S2. The co-culture method according to the first embodiment may further include a culture medium collection step S3a and a culture medium analysis step S4. The co-culture method according to the first embodiment may include a bacteria addition step S3b instead of culture medium collection step S3a.

In oxygen concentration adjustment step S1, the dissolved oxygen concentration in the first culture medium to be supplied to first flow path 11 is adjusted. More specifically, oxygen concentration adjustment step S1 is performed by passing the first culture medium through third flow path 31 and passing the third culture medium through fourth flow path 32. During the flow of the first culture medium through third flow path 31, the bacteria in the first culture medium and the cells cultured on third main surface 33a consume oxygen, and hence the dissolved oxygen concentration in the first culture medium that has flowed through third flow path 31 (that is, the first culture medium to be supplied to first flow path 11) is adjusted. In oxygen concentration adjustment step S1, during the flow of the third culture medium through fourth flow path 32, oxygen in the third culture medium is supplied toward third flow path 31 through second membrane 33. The third culture medium that has flowed through fourth flow path 32 is subjected to culture medium analysis step S4.

In culture medium treatment step S2, the first culture medium supplied to first flow path 11 and the second culture medium supplied to second low path 12 are treated. More specifically, culture medium treatment step S2 is performed by passing the first culture medium through first flow path 11 and passing the second colure medium through the second flow path. In culture medium treatment step S2, during the flow of the second culture medium through second flow path 12, oxygen in the second culture medium is supplied toward first flow path 11 through first membrane 13. The first culture medium that has flowed through first flow path 11 is subjected to culture medium analysis step S4.

In culture medium collection step S3a, the first culture medium that has flowed through third flow path 31 is partially collected before being supplied to first flow path 11. This collection is carried out by the septum connected to third connection port 40c of three-way connector 40. The first culture medium collected in culture medium collection step S3a is partially subjected to culture medium analysis step S4.

In bacteria addition step S3b, bacteria are added to the first culture medium that has flowed through third flow path 31 before the first culture medium is supplied to first flow path 11. The bacteria added in bacteria addition step S3b may be the same as or different from the bacteria contained in the first culture medium flowing through third flow path 31.

In culture medium analysis step S4, mass spectrometry is performed on the first culture medium that has flowed through first flow path 11, the second culture medium that has flowed through second flow path 12, and the third culture medium that has flowed through fourth flow path 32. For example, liquid chromatography mass spectrometry is employed as this mass spectrometry. Prior to this mass spectrometry, desired pretreatment may be performed on the first culture medium that has flowed through first flow path 11, the second culture medium that has flowed through second flow path 12, and the third culture medium that has flowed through fourth flow path 32.

Note that culture medium analysis step S4 should only be performed on at least one of the first culture medium that has flowed through first flow path 11, the second culture medium that has flowed through second flow path 12, and the third culture medium that has flowed through fourth flow path 32. Culture medium analysis step S4 may be performed on the first culture medium collected in culture medium collection step S3a.

Effects of co-culture device 100 and the co-culture method according to the first embodiment will be described below.

In co-culture device 100, the dissolved oxygen concentration in the first culture medium to be supplied to first flow path 11 can be adjusted by oxygen concentration adjuster 20 (second body 30). According to co-culture device 100, therefore, an environment having different oxygen concentrations such as one from the small intestine to the large intestine can be simulated.

When first body 10 and second body 30 are immersed in the liquid stored in container 50 in co-culture device 100, mixing of oxygen into the culture media flowing through co-culture device 100 can be suppressed. When co-culture device 100 includes degasser 52, mixing of oxygen into the culture media flowing through co-culture device 100 can be further suppressed. When co-culture device 100 includes heater 51, effect of temperature outside of co-culture device 100 on the culture medium treatment in co-culture device 100 can be suppressed.

When co-culture device 100 includes three-way connector 40, bacteria can be added to the first culture medium that has flowed through third flow path 31. Therefore, an environment having not only different oxygen concentrations but also different types of bacteria and/or different amounts of bacteria can be simulated. When co-culture device 100 includes three-way connector 40, the first culture medium that has flowed through third flow path 31 can partially be collected before being supplied to first flow path 11. Therefor, a process of changing environment can be monitored.

A state of the cells cultured on first main surface 13a (such as whether the tight junctions have been lost or not) manifests itself in variation in the electrical resistance value between first flow path 11 and second flow path 12. Thus, when first body 10 includes electrode 16a and electrode 16b, the state of the cells cultured on first main surface 13a can be monitored.

According to the co-culture method in the first embodiment, the dissolved oxygen concentration in the first culture medium to be supplied to first flow path 11 is adjusted. Therefore, an environment having different oxygen concentrations can be simulated.

When the first culture medium that has flowed through third flow path 31 is partially collected before being supplied to first flow path 11 in the co-culture method according to the first embodiment, a process of changing environment can be monitored. When bacteria are added to the first culture medium that has flowed through third flow path 31 before the first culture medium is supplied to first flow path 11 in the treatment method according to the first embodiment, an environment having not only different oxygen concentrations but also different types of bacteria and/or different amounts of bacteria can be simulated.

When mass spectrometry is performed on the first culture medium that has flowed through first flow path 11, the second culture medium that has flowed through the second flow path, the first culture medium that has flowed through the third flow path, and the third culture medium that has flowed through the fourth flow path in the co-culture method according to the first embodiment, an activity condition of the bacteria contained in the first culture medium, or absorption of the bacteria contained in the first culture medium into cells of a metabolite, for example, can be analyzed.

### Second Embodiment

The configuration of a co-culture device according to a second embodiment (hereinafter referred to as “co-culture device 200”) is described below. Differences from the configuration of co-culture device 100 will mainly be described here, and redundant description will not be repeated.

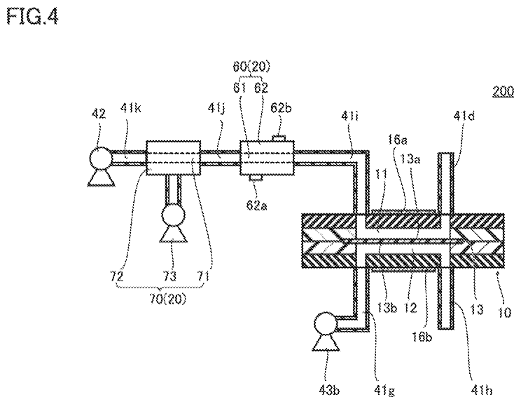

FIG. 4 is a schematic cross-sectional view of co-culture device 200. As shown in FIG. 4, co-culture device 200 includes first body 10 and oxygen concentration adjuster 20. In co-culture device 200, the dissolved oxygen concentration in the first culture medium to be supplied to first flow path 11 is adjusted by oxygen concentration adjuster 20. In these respects, co-culture device 200 is similar in configuration to co-culture device 100.

However, co-culture device 200 is different in configuration from co-culture device 100 in the configuration of oxygen concentration adjuster 20. In co-culture device 200, oxygen concentration adjuster 20 is formed of a gas exchanger 60 and a degasser 70. In co-culture device 200, oxygen concentration adjuster 20 may be formed only of gas exchanger 60 or only of degasser 70.

Gas exchanger 60 has a tube 61 and a chamber 62, for example. Tube 61 has an outlet connected to a tube 41i which is connected to first flow path 11. Tube 61 is made of a material having high gas permeability. Tube 61 is disposed within chamber 62. Tube 61 has an inlet connected to a tube 41j. By driving pump 42, the first culture medium flows through tube 41j to tube 61.

Chamber 62 has a gas inlet 62a and a gas outlet 62b. Gas inlet 62a and gas outlet 62b communicate with the interior of chamber 62. Through gas inlet 62a, atmospheric gas is supplied into chamber 62. This atmospheric gas is a gas not containing oxygen. For example, this atmospheric gas is a gas composed of 5 volume percent of carbon dioxide and nitrogen forming the remainder.

The atmospheric gas supplied into chamber 62 through gas inlet 62a exchanges gas with the first culture medium flowing through tube 61. The dissolved oxygen concentration in the first culture medium flowing through tube 61 thereby decreases. As a result, the first culture medium having the adjusted dissolved oxygen concentration is supplied to first flow path 11. The atmospheric gas that has exchanged gas with the first culture medium flowing through tube 61 is discharged from the interior of chamber 62 through gas outlet 62b.

Degasser 70 includes a tube 71, a chamber 72, and a pump 73, for example. Tube 71 is made of a material having high gas permeability. Tube 71 has an outlet connected to tube 41j. Tube 71 has an inlet connected to a tube 41k. By driving pump 42, the first culture medium is supplied through tube 41k to tube 71. Tube 71 is disposed within chamber 72.

By driving pump 73, the interior of chamber 72 is evacuated. Thus, oxygen in the first culture medium flowing through tube 71 is released through tube 71 into chamber 72, and the first culture medium flowing through tube 71 is degassed. The dissolved oxygen concentration in the first culture medium to be supplied to first flow path 11 is adjusted also by this degassing.

Note that tube 41i, tube 41j and tube 41k are made of a material having low gas permeability (for example, PEEK resin).

Though the example above has been described with respect to the case where there is one first body 10, there may be two or more first bodies 10. When there are two or more first bodies 10, first body 10 other than first body 10 connected to second body 30 is connected in series. One first body 10 connected to the upstream side of another first body 10 functions as oxygen concentration adjuster 20 for the another first body 10 connected to the downstream side of the one first body 10.

A co-culture method according to the second embodiment will be described below. Differences from the co-culture method according to the first embodiment will mainly be described here, and redundant description will not be repeated.

As with the co-culture method according to the first embodiment, the co-culture method according to the second embodiment includes oxygen concentration adjustment step S1, culture medium treatment step S2, and culture medium analysis step S4.

However, the co-culture method according to the second embodiment is different from oxygen concentration adjustment step S1 according to the first embodiment in that oxygen concentration adjustment step S1 is performed by gas exchange with the first culture medium to be supplied to first flow path 11. Oxygen concentration adjustment step S1 in the co-culture method according to the second embodiment may be performed by degassing of the first culture medium to be supplied to first flow path 11.

Effects of co-culture device 200 and the co-culture method according to the second embodiment will be described below. Differences from the effects of co-culture device 100 and the co-culture method according to the first embodiment will mainly be described here, and redundant description will not be repeated.

In co-culture device 200, the dissolved oxygen concentration in the first culture medium to be supplied to first flow path 11 can be adjusted by gas exchanger 60 and/or degasser 70 serving as oxygen concentration adjuster 20. According to co-culture device 200, therefore, an environment having different oxygen concentrations such as one from the small intestine to the large intestine can be simulated.

According to the co-culture method in the second embodiment, the dissolved oxygen concentration in the first culture medium to be supplied to first flow path 11 is adjusted in a manner similar to the co-culture method according to the first embodiment. Therefore, an environment having different oxygen concentrations can be simulated.

Though the embodiments of the present invention have been described as above, various modifications to the embodiments described above are possible. In addition, the scope of the present invention is not limited to the embodiments described above. The scope of the present invention is defined by the terms of the claims, and is intended to include any modifications within the meaning and scope equivalent to the terms of the claims.

Though embodiments of the present invention have been described, it should be understood that the embodiments disclosed herein are illustrative and non-restrictive in every respect. The scope of the present invention is defined by the terms of the claims and is intended to include any modifications within the meaning and scope equivalent to the terms of the claims.

What is claimed is:

1. A co-culture device comprising:
a first body including
a first membrane having a first main surface for culturing cells and a second main surface opposite to the first main surface,
a first flow path partially defined by the first main surface, the first flow path configured for a first culture medium to flow therethrough, and
a second flow path partially defined by the second main surface, the second flow path configured for a second culture medium having a higher dissolved oxygen concentration than that of the first culture medium to flow therethrough; and
an oxygen concentration adjuster for adjusting the dissolved oxygen concentration in the first culture medium to be supplied to the first flow path.

2. The co-culture device according to claim 1, wherein
the oxygen concentration adjuster includes a tube through which the first culture medium to be supplied to the first flow path flows, and a gas exchanger in which the first culture medium flowing through the tube exchanges gas with atmospheric gas around the tube.

3. The co-culture device according to claim 1, wherein
the oxygen concentration adjuster is a second body including
a second membrane having a third main surface for culturing cells and a fourth main surface opposite to the third main surface,
a third flow path partially defined by the third main surface, the third flow path configured for the first culture medium to be supplied to the first flow path to flow therethrough, and
a fourth flow path partially defined by the fourth main surface, the fourth flow path configured for a third culture medium having a higher dissolved oxygen concentration than that of the first culture medium flowing through the third flow path to flow therethrough.

4. The co-culture device according to claim 3, further comprising a three-way connector having a first connection port, a second connection port, and a third connection port to which a septum can be connected, wherein
the first culture medium that has lowed through the third flow path flows into the three-way connector through the first connection port, and flows out through the second connection port and is supplied to the first flow path.

5. The co-culture device according to claim 3, further comprising a container that stores liquid in which the first body and the second body are immersed.

6. The co-culture device according to claim 5, further comprising a heater that maintains a temperature of the liquid.

7. The co-culture device according to claim 5, further comprising a degasser that degases oxygen in the liquid.

8. The co-culture device according to claim 1, wherein
the first body further includes an electrode capable of measuring an electric resistance between the first flow path and the second flow path.

9. A co-culture method comprising:
adjusting a dissolved oxygen concentration in a first culture medium;
passing the first culture medium having the adjusted dissolved oxygen concentration through a first flow path partially defined by a first main surface of a first membrane on which cells are cultured; and
passing a second culture medium having a higher dissolved oxygen concentration than that of the first culture medium through a second flow path partially defined by a second main surface of the first membrane opposite to the first main surface, and supplying oxygen from the second culture medium toward the first flow path through the first membrane.

10. The co-culture method according to claim 9, wherein
the adjusting the dissolved oxygen concentration in the first culture medium includes
passing the first culture medium through a third flow path partially defined by a third main surface of a second membrane on which cells are cultured, and
passing a third culture medium having a higher dissolved oxygen concentration than that of the first culture medium through a fourth flow path partially defined by a fourth main surface of the second membrane opposite to the third main surface, and supplying oxygen from the third culture medium toward the third flow path through the second membrane.

11. The co-culture method according to claim 10, further comprising adding bacteria to the first culture medium after the first culture medium flows through the third flow path and before the first culture medium flows through the first flow path.

12. The co-culture method according to claim 10, further comprising partially collecting the first culture medium after the first culture medium flows through the third flow path and before the first culture medium flows through the first flow path.

13. The co-culture method according to claim 9, wherein
the adjusting the dissolved oxygen concentration in the first culture medium is performed by gas exchange with the first culture medium.

14. The co-culture method according to claim 9, further comprising performing mass spectrometry on at least one of the first culture medium that has flowed through the first flow path and the second culture medium that has flowed through the second flow path.
